(19) United States
(12) Patent Application Publication
(10) Pub. No.: US 2026/0131126 A1
(43) Pub. Date: May 14, 2026

(54) INTEGRATED FILL TUBE FOR AN INTRODUCER SHEATH ASSEMBLY

(71) Applicant: W. L. Gore & Associates, Inc., Newark, DE (US)

(72) Inventor: David J. Arcaro, Flagstaff, AZ (US)

(21) Appl. No.: 19/385,334

(22) Filed: Nov. 11, 2025

Related U.S. Application Data
(60) Provisional application No. 63/719,396, filed on Nov. 12, 2024.

(51) Int. Cl. A61M 39/06; A61M 25/06

(52) U.S. Cl. CPC A61M 39/0613; A61M 25/0662; A61M 2025/0681; A61M 2039/062

(57) ABSTRACT

An introducer sheath comprising an outer tube extending along a longitudinal axis defining an interior surface and an exterior surface, and an interior volume defined inside the interior surface, the outer tube including an outer tube first end, an outer tube second end opposite the outer tube first end, a fill tube integrally formed with the exterior surface of the outer tube, the fill tube positioned intermediate the outer tube first end and the outer tube second end, the fill tube extending outwardly from the exterior surface of the outer tube and fluidly coupled to the interior volume, and an outer tube center portion positioned intermediate the outer tube first end and the outer tube second end, the outer tube center portion configured to radially deflect.

### CROSS-REFERENCE TO RELATED APPLICATION

This application claims the benefit of Provisional Application No. 63/719,396, filed Nov. 12, 2024, which is incorporated herein by reference in its entirety for all purposes.

### BACKGROUND

During transcatheter and orthoscopic procedures, an insertion site is created on a patient in order to gain access to the target tissue of the procedure. However, insertion sites also provide an opening in the skin from which fluids from the patient (e.g., blood) may flow out. Some procedures require larger insertion sites in order to advance larger tools or devices. This may result in situations in which an undesirable amount of fluid or blood is lost through the insertion site.

### SUMMARY

An introducer sheath assembly may include a pair of tube members radially separated to create a pressurizable space. One or more of the tube members may include a fill tube fluidly coupled to the pressurizable space. Fill tubes may be integrally formed with one of the tubes. Fill tubes and methods of forming the fill tubes are disclosed herein.

According to one example (“Example 1”), an introducer sheath comprises an outer tube extending along a longitudinal axis between an outer tube first end and an outer tube second end opposite the outer tube first end with an outer tube center portion therebetween, the outer tube defining an interior surface and an exterior surface, the interior surface defining an interior volume, the outer tube including a fill tube integrally formed with the exterior surface of the outer tube, the fill tube positioned intermediate the outer tube first end and the outer tube second end, the fill tube extending outwardly from the exterior surface of the outer tube and fluidly coupled to the interior volume, and the outer tube center portion formed of a flexible material and operable to distend relative to the longitudinal axis.

According to another example (“Example 2”), further to Example 1, an inner tube is positioned at least partially within the interior volume.

According to another example (“Example 3”), further to Example 2, the inner tube is formed of a drapeable material.

According to another example (“Example 4”), further to any of Examples 2-3, the inner tube is formed of an expanded polyethylene (ePE).

According to another example (“Example 5”), further to any of Examples 2-3, the inner tube is formed of an Aliphatic Thermoplastic Polyether Polyurethane (ATPU).

According to another example (“Example 6”), further to any of the preceding Examples, the fill tube is positioned adjacent the outer tube first end.

According to another example (“Example 7”), further to any of the preceding Examples, the fill tube is positioned adjacent the outer tube center portion.

According to another example (“Example 8”), further to any of the preceding Examples, the fill tube extends radially outwardly generally perpendicular to the longitudinal axis.

According to another example (“Example 9”), further to any of the preceding Examples, the tube and the fill tube are formed of a silicone material.

According to one example (“Example 10”), an introducer sheath assembly comprises an outer tube including an outer tube first end and an outer tube second end and a fill tube integrally formed and positioned intermediate the outer tube first end and the outer tube second end, an inner tube positioned within the outer tube, and a pressurizable space defined between the outer tube and the inner tube, the fill tube fluidly coupled with the pressurizable space.

According to another example (“Example 11”), further to Example 10, the introducer sheath assembly extends along a longitudinal axis, and the fill tube extends radially outwardly from the outer tube and perpendicular to the longitudinal axis.

According to another example (“Example 12”), further to any of Examples 10-11, a collar is coupled to each of the outer tube and the inner tube.

According to another example (“Example 13”), further to Example 12, the outer tube includes a recess, the inner tube includes a ring protrusion, and the ring protrusion received within the recess.

According to another example (“Example 14”) further to any of Examples 10-13, the fill tube is formed of the same material as the outer tube.

According to another example (“Example 15”), further to any of Examples 10-14, the outer tube is formed of an expandable material.

According to another example (“Example 16”), further to Example 15, each of the outer tube and the fill tube is formed of a silicone material.

According to another example (“Example 17”), further to any of Examples 10-16, the outer tube is formed of a flexible material.

According to another example (“Example 18”), further to any of Examples 10-17, the inner tube is formed of a drapeable material.

According to another example (“Example 19”), further to any of Examples 10-18, the inner tube is formed of an expanded polyethylene (ePE).

According to another example (“Example 20”), further to any of Examples 10-18, the inner tube is formed of an Aliphatic Thermoplastic Polyether Polyurethane (ATPU).

According to one example (“Example 21”), a method of manufacturing a medical device comprises forming an inner tube defining an inner tube first end portion, an inner tube second end portion, and an inner tube center portion, forming an outer tube extending along a longitudinal axis and defining an outer tube first end portion, an outer tube second end portion, an outer tube center portion, and a fill tube extending radially outwardly and generally perpendicular to the longitudinal axis, the fill tube being integrally formed with each of the outer tube first end portion, the outer tube second end portion, and the outer tube center portion, positioning the inner tube first end portion adjacent the outer tube first end portion, positioning the inner tube second end portion adjacent the outer tube second end portion, coupling a first collar to each of the inner tube first end portion and the outer tube first end portion, and coupling a second collar to each of the inner tube second end portion and the outer tube second end portion to create a pressurizable space intermediate the outer tube and the inner tube, the fill tube being fluidly coupled with the pressurizable space.

According to another example (“Example 22”), further to Example 21, the fill tube defines a fill tube aperture, the method further comprising coupling a valve to the fill tube aperture, the valve operable to selectively allow fluid to flow into the pressurizable space.

According to another example (“Example 23”), further to any of Examples 21-22, the outer tube is formed of a flexible material.

According to another example (“Example 24”), further to Example 23, the outer tube is formed of a silicon material.

According to another example (“Example 25”), further to any of Examples 21-24, the outer tube includes an extension positioned adjacent the outer tube first end portion.

According to another example (“Example 26”), further to any of Examples 21-25, the fill tube is positioned adjacent the outer tube first end portion.

According to another example (“Example 27”), further to any of Examples 21-26, the fill tube is positioned adjacent the outer tube central portion.

According to another example (“Example 28”), further to any of Examples 21-27, the fill tube includes an aperture, and the outer tube defines a first length between the outer tube first end portion and the outer tube second end portion, and the fill tube defines a second length between an exterior surface of the outer tube and the aperture, and the second length is smaller than the first length.

According to another example (“Example 29”), further to any of Examples 21-28, the outer tube is formed by a liquid injection molding process.

According to another example (“Example 30”), further to any of Examples 21-29, the inner tube is formed of a drapeable material.

According to another example (“Example 31”), further to any of Examples 21-30, the inner tube is formed of an expanded polyethylene (ePE).

According to another example (“Example 32”), further to any of Examples 21-30, the inner tube is formed of an Aliphatic Thermoplastic Polyether Polyurethane (ATPU).

The foregoing Examples are just that, and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a perspective view of an introducer sheath valve assembly in accordance with an embodiment of the present disclosure;

FIG. 2 is a side view of the introducer sheath valve assembly of FIG. 1 in accordance with an embodiment of the present disclosure;

FIG. 3 is an exploded view of an introducer sheath valve assembly in accordance with an embodiment of the present disclosure;

FIG. 4 is a section view of an introducer sheath valve assembly in accordance with an embodiment of the present disclosure;

FIG. 5 is a side view of an inner tube member of an introducer sheath valve assembly in accordance with an embodiment of the present disclosure;

FIG. 6 is an end-on view of an introducer sheath valve assembly in accordance with an embodiment of the present disclosure;

FIG. 7A is a perspective view of an outer tube of an introducer sheath valve assembly in accordance with an embodiment of the present disclosure;

FIG. 7B is a perspective view of an outer tube of an introducer sheath valve assembly in accordance with an embodiment of the present disclosure;

FIG. 8 is an exploded view of an outer tube of an introducer sheath assembly in accordance with an embodiment of the present disclosure;

FIG. 9 is a perspective view of a collar of an introducer sheath valve assembly in accordance with an embodiment of the present disclosure;

FIG. 10 is a section view of an outer tube of an introducer sheath valve assembly in accordance with an embodiment of the present disclosure; and

FIG. 11 is a perspective view of an outer tube of an introducer sheath assembly in accordance with an embodiment of the present disclosure.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably, to refer to a measurement that includes the stated measurement and that also includes any measurements that are reasonably close to the stated measurement. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

The term “drape” or “drapeable” can be understood to mean the way a fabric or a material falls when hung in different positions such as the ability of the fabric or material to conform to the shape of another object when laid upon it. Drape is associated with flexibility and suppleness of a fabric or a material.

### Description of Various Embodiments

Persons skilled in the art will readily appreciate that various aspects of the present disclosure can be realized by any number of methods and apparatuses configured to perform the intended functions. It should also be noted that the accompanying drawing figures referred to herein are not necessarily drawn to scale, but may be exaggerated to illustrate various aspects of the present disclosure, and in that regard, the drawing figures should not be construed as limiting.

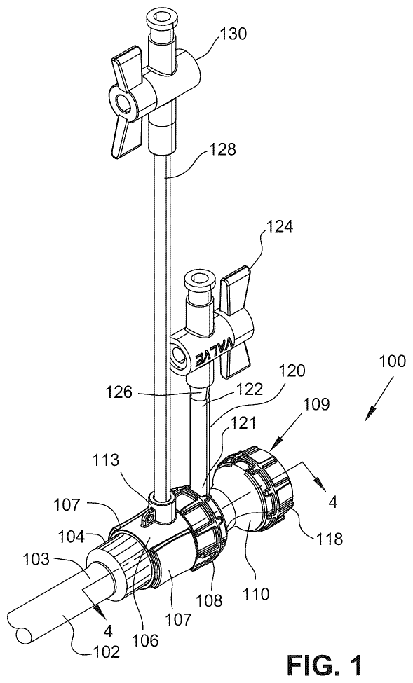

Referring to FIGS. 1-4, an introducer sheath valve assembly 100 (i.e., a medical device) for insertion of tools and devices during transcatheter procedures includes an introducer sheath 102. The introducer sheath valve assembly 100 is capable of limiting back bleeding through the introducer sheath valve assembly 100 during transcatheter procedures. The introducer sheath 102 may be formed of any material with suitable biocompatible and mechanical properties, for example, fluorinated ethylene propylene (FEP), expanded high density polyethylene (eHDP), or any other material with suitable biocompatible and mechanical properties. The introducer sheath 102 may be of any size including but not limited to from about 12 Fr to 26 Fr or any size therebetween. The introducer sheath valve assembly 100 includes a threaded adapter 104 on an introducer sheath first end 103 of the introducer sheath 102. The threaded adapter 104 may be formed of any biocompatible plastic or any biocompatible metal with suitable biocompatible and mechanical properties. The threaded adapter 104 may be coupled to the introducer sheath 102 by a variety of means which may include, but are not limited to, adhesives such as polyurethane adhesives, quick setting cyanoacrylate adhesives, or ultraviolet cured adhesives, bonding such as thermal bonding, fusing, mechanical adapters, ultrasonic welding, by an interference fit, insert molding, and so forth.

Still referring to FIGS. 1-4, a first fitting 106 may be coupled to the second end of the threaded adapter 104. The first fitting 106 may include a profile that facilitates a user gripping the introducer sheath valve assembly 100 securely. The first fitting 106 may include one or more protrusions 107 to enhance traction for a user on the introducer sheath valve assembly 100. In some embodiments, the first fitting 106 may be formed with any biocompatible metal or plastic with suitable biocompatible and mechanical properties, and the protrusions 107 may be made of a similar material as the first fitting 106 or may be made of a material with a high coefficient of friction, a material more compliant than the first fitting 106, or made with grating, a roughening, a raised company logo or design, or striations in the surface in conjunction with the material listed above to further aid in the gripping of the device. These features on the surface of the first fitting 106 may also be used to aid in gripping without the use of the protrusions 107 and may be applied directly to a lateral surface (e.g., a gripping or contact surface) of the first fitting 106. In some embodiments, a flush port 113 may extend from the first fitting 106 (e.g., outwardly from a lateral surface of the first fitting 106).

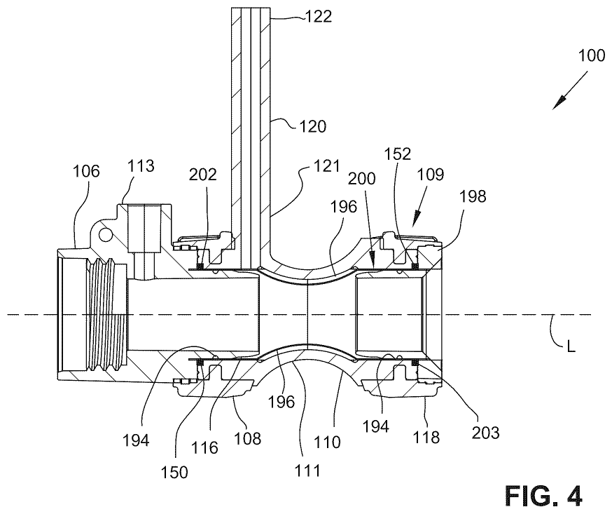

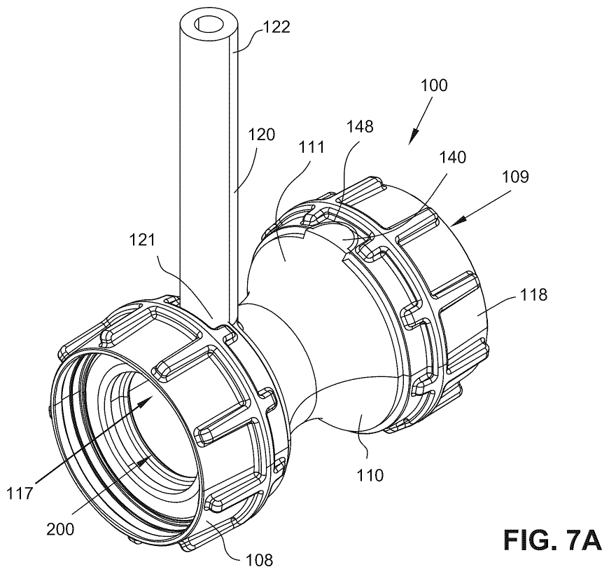

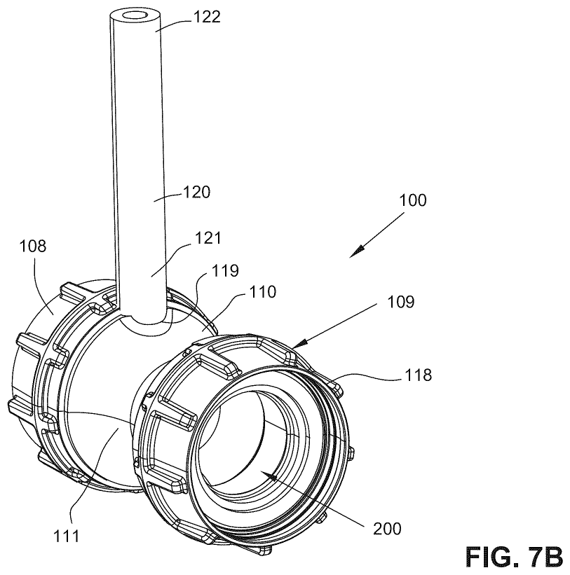

Still referring to FIGS. 1-4, the introducer sheath valve assembly 100 includes an outer tubular assembly 109. The outer tubular assembly 109 may include an outer tube 110. In some embodiments, the outer tubular assembly 109 may include a first collar 108 coupled to the first fitting 106 at an introducer sheath first end of the introducer sheath valve assembly 100, a second collar 118 positioned at a second end of the introducer sheath valve assembly 100, and the outer tube 110 coupled between the first collar 108 and the second collar 118. In some embodiments, the outer tube 110 extends along a longitudinal axis L and includes an outer tube center portion 111 defining an exterior surface 115 and an interior surface 112 defining an interior volume 117 (FIG. 7A). In embodiments, the outer tube center portion 111 is positioned intermediate an outer tube first end portion 133 and an outer tube second end portion 137. The first collar 108 includes a fill tube 120 including a fill tube first end 122 and a fill tube second end 121 opposite the fill tube first end 122. In some embodiments, the fill tube first end 122 defines a fill tube aperture 123. The fill tube 120 may be fluidly coupled with an interior of the outer tube 110 through the fill tube aperture 123. In some embodiments, each of the first collar 108, the second collar 118, and the fill tube 120 may be formed of any biocompatible metal or plastic with suitable biocompatible and mechanical properties. Optionally, the fill tube 120 may be located at any point along either of the first collar 108 or the second collar 118. Referring to FIG. 4, a second fitting 198 fits within the outer tube 110 and the second collar 114. Referring still to FIGS. 1-4, the fill tube 120 extends radially outwardly from an exterior surface of the outer tube 110. In some embodiments, the fill tube 120 extends radially outwardly from the outer tube second end portion 137 of the outer tube 110. In some embodiments, the fill tube 120 extends radially outwardly from the outer tube center portion 111 of the outer tube 110. In some embodiments, the fill tube 120 extends radially outwardly and generally perpendicular to the longitudinal axis L.

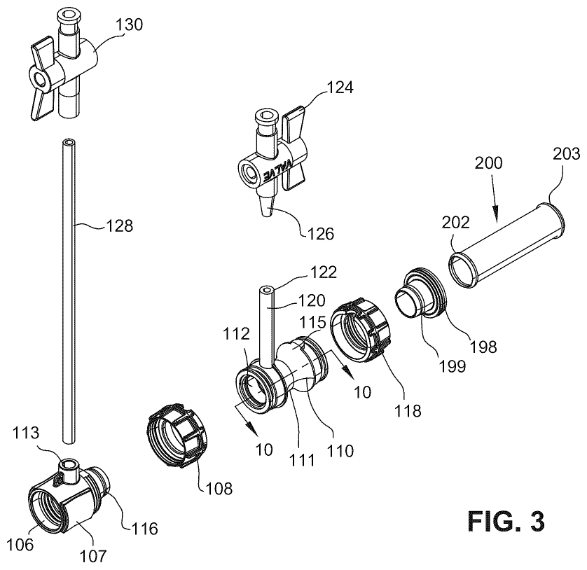

Referring now to FIGS. 3-5, the introducer sheath valve assembly 100 includes an inner tube 200 arranged inside the outer tube 110 and a pressurizable space 196 is defined between the outer tube 110 and the inner tube 200. The inner tube 200 (e.g., film tube) includes a pair of end rings including a first end ring 202 (e.g., a ring protrusion) positioned adjacent an inner tube first end portion and a second end ring 203 (e.g., a ring protrusion) positioned adjacent an inner tube second end portion. Referring to FIG. 4, the inner tube 200 is positioned and affixed between the outer tube 110 and the first fitting 106 at the first end ring 202 and between the outer tube 110 and the second fitting 198 at the second end ring 203. In some embodiments, inner tube 200 is formed of a flexible, thin, strong, drapeable material (e.g., a compliant material) such as expanded polyethylene (ePE), expanded polytetrafluoroethylene (ePTFE), FEP, Aliphatic Thermoplastic Polyether Polyurethane (ATPU), fabrics, silk, or Kevlar® brand fiber, and combinations thereof. These materials may be used as a single layer construct or a multi-layer construct, and may be formed as composite materials. For example, the inner tube 200 may be formed of a thin, porous polymeric substrate including multiple layers of material, which may be filled or imbibed with a secondary polymer. The secondary, filling or imbibing polymer may be the same as or similar to that of the construct or may be a different polymer.

Referring to FIG. 4, the outer tube 110 includes a pair of sealing lips 194 which may interface with each of the inner tube 200, the first fitting 106, and the second fitting 198. Additionally, the outer tube 110 includes a pair of recesses 150, 152 positioned at either end of the outer tube 110. The first end ring 202 of the inner tube 200 is sized and shaped to sit within the first recess 150 and the second end ring 203 of the inner tube 200 is sized and shaped to sit within the second recess 152. In some embodiments, each of the recesses 150, 152 and the end rings 202, 203 are sized and shaped such that the end rings 202, 203 are compressed within the recesses 150, 152.

Optionally, the outer tube 110 may be constructed of any material having desirable mechanical and biocompatible properties, including but not limited to any elastomer, latex, or polycarbonate with desirable mechanical and biocompatible properties. Optionally, the outer tube 110 is formed of any suitable expandable material. In embodiments, outer tube 110 is formed of a flexible material. In embodiments, a portion of outer tube 110 is formed of a flexible material. In embodiments, outer tube center portion 111 is formed of a flexible material. In one embodiment, the outer tube 110 comprises silicone and has an hourglass shape when not pressurized. When the pressurizable space 196 is pressurized to a sufficient pressure such that the inner tube 200 collapses, the outer tube center portion 111 of the outer tube 110 may subsequently elastically distend or radially deflects such that the hourglass shape of the outer tube 110 alters and provides indication of sufficient pressure in the pressurizable space 196 (e.g., when the pressurizable space 196 is pressurized with at least one substance to a sufficient pressure, the inner tube 200 collapses around a profile of any object positioned therethrough, for example, a tool or another endoluminal device, which limits back bleeding). This feature of the outer tube 110 facilitates the user of a device easily and quickly identifying the optimal pressure for the device.

FIGS. 3-5 illustrate the end rings 202, 203 on either ends of the inner tube 200. The end rings 202, 203 are used to create a stiff member to aid in attachment of the inner tube 200 to the first fitting 106 and to the second fitting 198. The end rings 202, 203 may be made of any material with desirable biocompatible and mechanical properties and will be described in greater detail below. The inner tube 200 with the attached end rings 202, 203 may be inserted through the outer tube 110 and attached to the protruding end 116 of the first fitting 106. The first collar 108 with the fill tube 120 may then be attached to the first fitting 106 with an adhesive, but other suitable attachment methods may suffice. The second end ring 203 may then be attached to the protrusion 199 on the second fitting 198. The second collar 118 may then be snapped onto the second fitting 198. That is, the first collar 108 may be coupled to the first end ring 202 and the first collar 108 may be used to mechanically constrain the first end ring 202 and the second collar 118 may be coupled to the second end ring 203 and the second collar 118 may be used to mechanically constrain the second end ring 203.

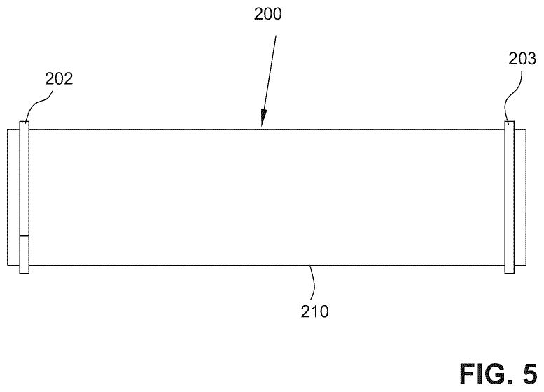

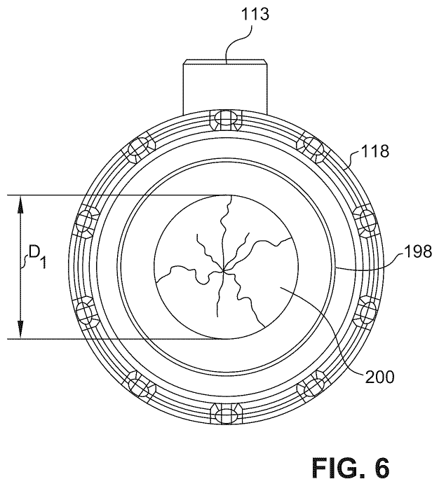

FIG. 6. Illustrates an end on view of the device showing an inner tube 200 with a diameter D1. The second collar 118 with the fill tube 120 (see FIG. 3) has a feature that allows the pressurizable space 196 (see FIG. 4) to be filled to a sufficient pressure to cause the inner tube 200 to collapse around a profile of any object positioned therethrough, for example, a tool or another endoluminal device (e.g., the fill tube 120 is fluidly coupled with the pressurizable space 196 to allow fluid to move into and out of the pressurizable space 196). The pressurizable space 196 may be filled with any suitable material or materials. For example, the pressurizable space 196 may be filled with one or more of the following substances: air, silicone, water, saline solution, low volatility biocompatible liquids, glycerin, propylene glycol, polyethylene glycol, compressible foam, elastomeric spheres, and crosslinked silicone gels. The inner tube 200 has an inner tube center portion 210 (see FIG. 5) that defines a first diameter D1 that varies with the pressure of the pressurizable space 196. That is, as the pressure within the pressurizable space 196 increases, the diameter D1 decreases such that the inner tube 200 constricts or transitions toward a collapsed configuration. As the inner tube 200 constricts or collapses, the inner tube 200 may contract and conform around a profile of (e.g., drape around) any object positioned therethrough, for example, a tool, to prevent back bleeding through the inner tube 200.

Referring again to FIGS. 1-3, a first valve 124 includes a valve end 126 operable to couple with the fill tube aperture 123 of the fill tube first end 122 of the fill tube 120. In some embodiments, the first valve 124 is operable to selectively open and close access to the fill tube 120 and the pressurizable space 196. In some embodiments, the introducer sheath valve assembly 100 includes a flush tube 128 fluidly coupled with the flush port 113. In some embodiments, a second valve 130 is operably coupled to the flush tube 128, and the second valve 130 is operable to open and close access to the flush tube 128. Optionally, each of the first valve 124 and the second valve 130 are stopcock valves.

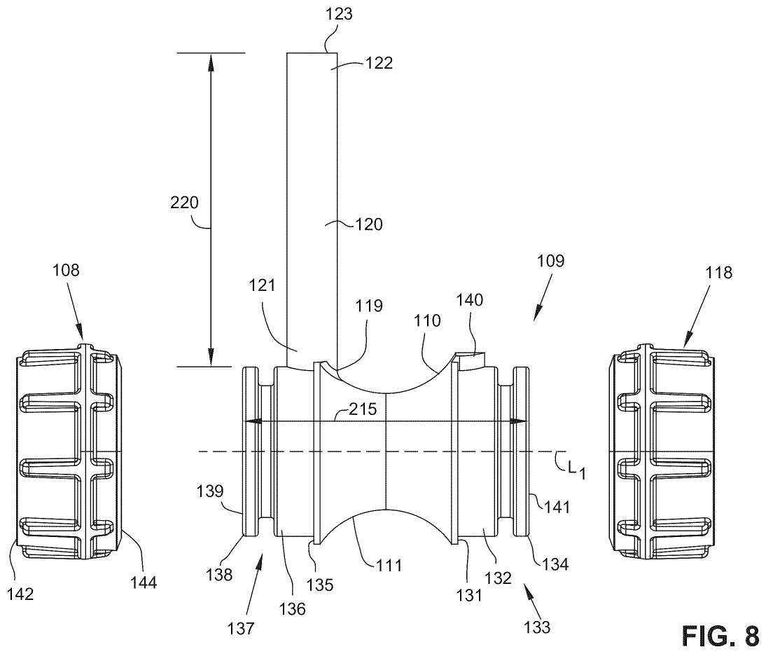

Referring now to FIGS. 7A-8, outer tubular assembly 109 includes the fill tube 120 integrally formed with the outer tube 110. That is, the fill tube 120 may be continuously formed (integrally, or monolithically as a single piece) with the outer tube 110. The outer tube 110 extends along the longitudinal axis L and includes an outer tube first end portion 133, an outer tube second end portion 137, and an outer tube center portion 111 positioned longitudinally intermediate the outer tube first end portion 133 and the outer tube second end portion 137. The outer tube center portion 111 defines an outer tube first axial end face 131 and an outer tube second axial end face 135. The outer tube first end portion 133 includes a first shoulder portion 132 extending axially outwardly from the outer tube first axial end face 131 of the outer tube center portion 111 and a first flange 134 extending axially outwardly from the first shoulder portion 132. The outer tube second end portion 137 includes a second shoulder portion 136 extending axially outwardly from the outer tube second axial end face 135 of the outer tube center portion 111 and a second flange 138 extending axially outwardly from the second shoulder portion 136. In some embodiments, the first collar 108 snaps onto the second flange 138 and the second collar 118 snaps onto the first flange 134.

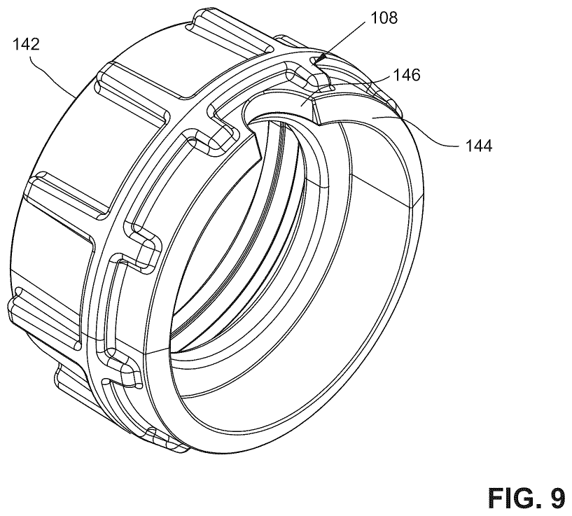

Referring to FIGS. 7A-9, the outer tube 110 includes a first extension 140 extending radially outwardly from the first shoulder portion 132. In some embodiments, the fill tube 120 is integrally coupled with the outer tube 110 adjacent the outer tube second axial end face 135. In some embodiments, the fill tube 120 is integrally coupled with the outer tube 110 adjacent the outer tube center portion 111. In some embodiments, the fill tube 120 is integrally coupled with the outer tube 110 adjacent the second shoulder portion 136. In some embodiments, the fill tube 120 is integrally coupled (e.g., integrally formed) with the outer tube 110 adjacent each of the second shoulder portion 136, the outer tube second axial end face 135, and the outer tube center portion 111. The fill tube second end 121 of the fill tube 120 may be integral with the outer tube 110 and a support or chamfer 119, extends around the fill tube second end 121 to strengthen the joint between the fill tube second end 121 of the fill tube 120 and the outer tube 110. In some embodiments, the chamfer 119 is an excess of material built up around the fill tube second end 121 of the fill tube 120 to provide support (e.g., from transverse forces) to the fill tube 120. Referring to FIGS. 8-9, first collar 108 includes a first collar first end 142 and a first collar second end 144 opposite the first collar first end 142, and the first collar second end 144 is facing generally inwardly toward the fill tube 120 and the outer tube center portion 111. In some embodiments, the first collar second end 144 includes a scalloped portion 146. In some embodiments, the second collar 118 includes a corresponding scalloped portion 148.

Referring to FIGS. 7A-7B, the first collar 108 couples with the outer tube second end portion 137 of the outer tube 110 such that the fill tube 120 sits within the scalloped portion 146 of the first collar 108 and the second collar 118 couples with the outer tube first axial end face 131 of the outer tube 110 such that the first extension 140 sits within the scalloped portion 148. That is, each of the fill tube 120 and the scalloped portion 146 as well as the first extension 140 and the scalloped portion 148 act as keying features for the first collar 108 and the second collar 118.

Referring again to FIG. 8, the outer tube 110 includes a length 215 defined between a first extent (e.g., an outer face 141 of the outer tube first end portion 133 of the outer tube 110) and a second extent (e.g., an outer face 139 of the outer tube second end portion 137 of the outer tube 110). Further, the fill tube 120 is dimensioned to have a length 220 defined between an outer surface of the second shoulder portion 136 and the fill tube extent (e.g., the adjacent the fill tube aperture 123). The length 220 may be approximately similar to the length 215. In some embodiments, the length 220 is greater than the length 215. In some embodiments, the length 220 is smaller than the length 215.

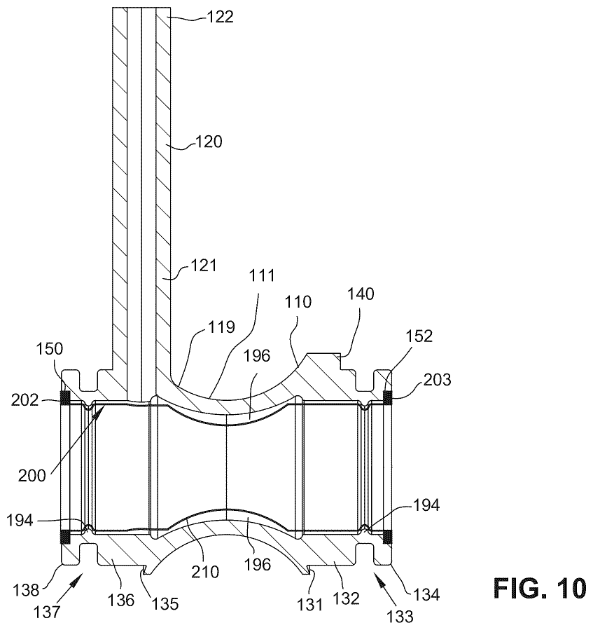

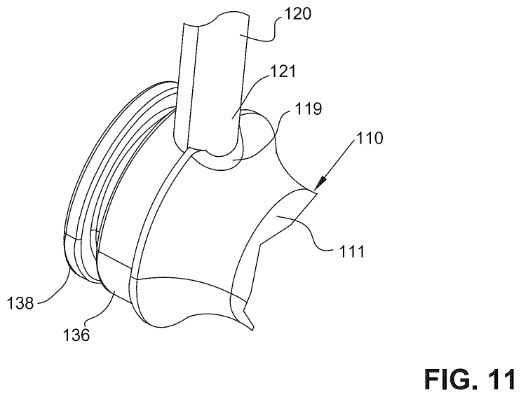

Referring to FIGS. 10-11, the outer tube 110 (e.g., the outer tube center portion 111, the first shoulder portion 132, the first flange 134, the second shoulder portion 136, the second flange 138, and the fill tube 120) is formed of a single, uniform material. Optionally, the outer tube 110 is formed of a silicone material. Optionally, the outer tube 110 is formed by an injection molding process. Optionally, the outer tube 110 is formed by a liquid injection molding process. In some embodiments, the outer tube 110 may be formed of any suitable material with suitable mechanical and biocompatible characteristics. In some embodiments, the outer tube 110 may be formed of any suitable material that allows for unitary construction/manufacture and retains suitable compliancy post-manufacture to allow for proper distension of the outer tube 110 when the pressurizable space 196 is under pressure. Optionally, the outer tube 110 may be formed of a silicone material and subsequently treated with a post cure (e.g., with a UV light) to ensure consistent properties throughout the silicone of the outer tube 110.

In some embodiments, leaks within the introducer sheath valve assembly 100 may be reduced and the strength of the fill tube 120 and the outer tube 110 may be increased by the fill tube 120 being integral with the remaining portions of the outer tube 110. Additionally, the reliability of the outer tube 110 and the introducer sheath valve assembly 100 may be increased by reducing the overall part count. That is, in one example, a continuous surface and integral body is created between the outer tube center portion 111 and the fill tube 120, thereby reducing opportunities for leaking fluids.

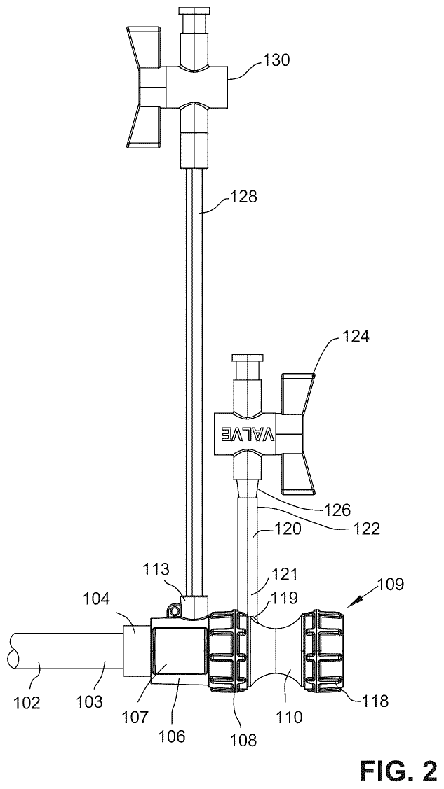

Referring again to FIGS. 1-2, the fill tube first end 122 of the fill tube 120 may be sufficiently compliant to stretch or expand and extend over the valve end 126 of the first valve 124. That is, the fill tube 120 may be fluidly coupled to the first valve 124 by an interference fit between the fill tube first end 122 of the fill tube 120 and the valve end 126 of the first valve 124. That is, the pressurizable space 196, the fill tube 120, and the first valve 124 may all be fluidly coupled to one another.

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

What is claimed is:

1. An introducer sheath comprising:
an outer tube extending along a longitudinal axis between an outer tube first end and an outer tube second end opposite the outer tube first end with an outer tube center portion therebetween, the outer tube defining an interior surface and an exterior surface, the interior surface defining an interior volume, the outer tube including:
a fill tube integrally formed with the exterior surface of the outer tube, the fill tube positioned intermediate the outer tube first end and the outer tube second end, the fill tube extending outwardly from the exterior surface of the outer tube and fluidly coupled to the interior volume, and
the outer tube center portion formed of a flexible material and operable to distend relative to the longitudinal axis.

2. The introducer sheath of claim 1, further comprising an inner tube positioned at least partially within the interior volume.

3. The introducer sheath of claim 2, wherein the inner tube is formed of a drapeable material.

4. The introducer sheath of claim 1, wherein the fill tube is positioned adjacent the outer tube first end.

5. The introducer sheath of claim 1, wherein the fill tube is positioned adjacent the outer tube center portion.

6. The introducer sheath of claim 1, wherein the fill tube extends radially outwardly generally perpendicular to the longitudinal axis.

7. The introducer sheath of claim 1, wherein the outer tube and the fill tube are formed of a silicone material.

8. An introducer sheath valve assembly, comprising:
an outer tube including an outer tube first end and an outer tube second end and a fill tube integrally formed and positioned intermediate the outer tube first end and the outer tube second end;
an inner tube positioned within the outer tube; and
a pressurizable space defined between the outer tube and the inner tube, the fill tube fluidly coupled with the pressurizable space.

9. The introducer sheath valve assembly of claim 8, wherein the introducer sheath valve assembly extends along a longitudinal axis, and the fill tube extends radially outwardly from the outer tube and perpendicular to the longitudinal axis.

10. The introducer sheath valve assembly of claim 8, further comprising a collar coupled to each of the outer tube and the inner tube.

11. The introducer sheath valve assembly of claim 10, wherein the outer tube includes a recess, the inner tube includes a ring protrusion, the ring protrusion received within the recess.

12. The introducer sheath valve assembly of claim 8, wherein the fill tube is formed of the same material as the outer tube.

13. The introducer sheath valve assembly of claim 8, wherein the outer tube is formed of an expandable material and each of the outer tube and the fill tube are formed of a silicone material.

14. The introducer sheath valve assembly of claim 8, wherein the outer tube is formed of a flexible material.

15. The introducer sheath valve assembly of claim 8, wherein the inner tube is formed of a drapeable material.

16. A method of manufacturing a medical device, comprising:
forming an inner tube defining an inner tube first end portion, an inner tube second end portion, and an inner tube center portion;
forming an outer tube extending along a longitudinal axis and defining an outer tube first end portion, an outer tube second end portion, an outer tube center portion, and a fill tube extending radially outwardly and generally perpendicular to the longitudinal axis, the fill tube being integrally formed with each of the outer tube first end portion, the outer tube second end portion, and the outer tube center portion;
positioning the inner tube first end portion adjacent the outer tube first end portion;
positioning the inner tube second end portion adjacent the outer tube second end portion;
coupling a first collar to each of the inner tube first end portion and the outer tube first end portion; and
coupling a second collar to each of the inner tube second end portion and the outer tube second end portion to create a pressurizable space intermediate the outer tube and the inner tube, the fill tube being fluidly coupled with the pressurizable space.

17. The method of claim 16, wherein the outer tube is formed of a silicone material.

18. The method of claim 16, wherein the outer tube includes an extension positioned adjacent the outer tube first end portion.

19. The method of claim 16, wherein the fill tube is positioned adjacent the outer tube first end portion.

20. The method of claim 16, wherein the fill tube includes an aperture, and the outer tube defines a first length between the outer tube first end portion and the outer tube second end portion, and the fill tube defines a second length between an exterior surface of the outer tube and the aperture, and the second length is smaller than the first length.

21. The method of claim 16, wherein the outer tube is formed by a liquid injection molding process.
